(19) United States
(12) Patent Application Publication
(10) Pub. No.: US 2008/0228168 A1
(43) Pub. Date: Sep. 18, 2008

(54) CATHETER WITH CHANGING MATERIAL PROPERTIES

(76) Inventors: Stephan Mittermeyer, Landshut (DE); Andreas Hartlep, Holzwirchen (DE)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 12/048,919

(22) Filed: Mar. 14, 2008

Related U.S. Application Data
(60) Provisional application No. 60/908,514, filed on Mar. 28, 2007.

(30) Foreign Application Priority Data
Mar. 16, 2007 (EP) 07005471

(51) Int. Cl. A61M 25/01

(52) U.S. Cl. 604/525

(57) ABSTRACT

A catheter for administering a substance into a patient's tissue includes an elongated catheter body surrounding a lumen. At least one part of the length of the catheter body includes a material that changes its stiffness due to changes in the ambient conditions in the administering environment.

### RELATED APPLICATION DATA

This application claims priority of U.S. Provisional Application No. 60/908,514 filed on Mar. 28, 2007, and EP 07005471 filed on Mar. 16, 2007, which are incorporated herein by reference in their entirety.

### FIELD OF THE INVENTION

The invention relates to a catheter for administering a substance into a body tissue. Such catheters may be introduced through the cranium into the brain tissue in neurosurgical procedures to release a substance directly in the brain tissue.

### BACKGROUND OF THE INVENTION

To achieve a reliable and predictable dispersion (in a patient) of a substance over a long period of time, for example several days, a catheter should be flexible once it has been placed. The flexibility allows the catheter to follow movements of the administering environment, for example movements of a brain. The flexibility also helps to ensure a homogenous interface between an outer surface of the catheter and the brain tissue. Once inserted, flexible catheters also may exhibit a lower backflow than rigid catheters, partly because they can adapt to movements of the brain (e.g., brain shift).

On the other hand, the catheter should be as rigid as possible during the placement, to allow the catheter to be stereotactically inserted with a high degree of precision, e.g., accurately positioned. Flexible catheters are conventionally inserted into the patient with the aid of a stylet made of a rigid material, for example metal. The combination provides a catheter that can be stereotactically exactly placed along a planned trajectory, and once the catheter has been placed, the stylet is removed and the catheter is secured to the scalp.

The flexible catheter with a stylet has at least two disadvantages. First, it requires a multi-part set of instruments. Second, the probability that air will enter the lumen of the catheter as the stylet is removed, is higher than that of a conventional catheter. When air is present in the lumen, introducing the liquid substance through the catheter also introduces this air into the tissue. When air bubbles present in the catheter are conveyed into the tissue, pressure peaks can be created during the infusion resulting from the compressibility of the air in the infusion line (lumen). The end result can be unreliable and unpredictable fluid dispersion patterns. The air bubbles can accumulate in the tissue or can travel along flow pathways in the tissue or can establish new flow pathways themselves. The air bubbles can amplify the backflow of the fluid along the outer surface of the catheter, also causing an inefficient and unpredictable dispersion of the fluid. Many treatments may be simulated on a computer. If air bubbles are introduced during the actual treatment, the likelihood of repeated the simulated dispersion of the substance is reduced.

A heating catheter having a variable stiffness is disclosed in U.S. Pat. No. 7,066,931. To make particular regions of the catheter more flexible, U.S. Pat. No. 7,066,931 proposes introducing openings, e.g., notches, slits, channels, grooves or holes, into the material of the catheter in these regions.

### SUMMARY OF THE INVENTION

A catheter for administering a substance into a body tissue (including brain structures) and can be placed without a stylet includes an elongated catheter body surrounding a lumen. At least one part of the length of the catheter body includes a material that can change its stiffness in response to changes in the ambient conditions in an administering environment.

In other words, the catheter can adapt its flexibility to its ambient conditions in a desirable, predictable way. When the catheter is placed in an administering environment, it can use the changes in the environment to change the stiffness properties of the catheter. The catheter may be rigid enough to follow a planned trajectory when being inserted (without a stylet), and, due a reduction in stiffness after placement, the catheter is flexible enough to follow movements of the tissue to ensure a predictable and reliable administration of the drug

An advantage using a catheter without a stylet is that smaller diameter catheters can be used. The smaller diameter reduces tissue trauma as well as help to reduce the backflow of fluid along the catheter.

Without need of a stylet, the catheter can be filled with the infusion fluid beforehand; this is referred to as priming. A catheter primed in this way does not introduce air bubbles into the tissue by subsequently flowing fluid, and the dispersion of the fluid is more reliable and more predictable.

The catheter material that changes its stiffness can be a material that is responsive to physical or chemical influencing factors. The physical or chemical influencing factors may include one or more of the following: changes in voltage and/or electrical current; magnetic field changes; pH values; temperature; water concentration; ion concentration; a concentration of a chemical substance or compound; a bodily ambient condition in the administering environment; or a property of the substance to be administered.

The factors for respectively changing the stiffness can be suitably selected depending on the instrumentation and/or ambient conditions present at the insertion location and/or administration location. The steric properties of the material may be altered, in particular the physical or chemical properties, wherein the alteration is triggered by a predictable or controllable influence in the specified location. One example of controlling the stiffness using a concentration of water is the use of hydrogels, such as silicone hydrogels or other stimuli-responsive hydro-gels.

Ambient conditions in the administration environment can have several meanings. The stiffness of the material can be changed by “external influences,” i.e. by influences that act on the catheter from outside the catheter. The stiffness of the material of the catheter can be changed from the inside using “internal influences.” An example can be an effect that the substance to be administered exerts on the material of the catheter when it flows in the lumen of the catheter at a certain flow rate. Combinations of such external and internal influences also are possible. One example of a combination could be external conditions (for example, a concentration of water or a concentration of ions) in the administration environment that provide preconditions for the change in stiffness, but the change in stiffness is only initiated when the substance also flows in the lumen and exerts an additional effect and/or serves as a catalyst. In this manner, a control mechanism can be used that only changes the stiffness during substance administration. An example of such a catheter can include materials based on a rubbery host polymer and rigid cellulose nanofibers.

The catheter can comprise an integrally formed catheter body comprising several materials, including an inner core that encloses the lumen, and an outer covering that surrounds the core. The catheter also can include of a single material that satisfies the conditions for changing the stiffness. An example of the catheter can be made of any uni- or multi-directionally oriented fibrous composite material.

If a covering/core configuration is selected, the covering can include a material that changes its stiffness when external conditions are altered, while the core includes a material that does not change its stiffness and insulates the covering from the influence of the substance to be administered. In this context, the inner surface of the catheter tube can be provided with a coating including a protective component, for example PTFE (polytetrafluoroethylene or Teflon), etc.

The opposite configuration can also be selected, in which a core includes a material that changes its stiffness in the presence of the substance to be administered, while the covering includes a material that does not change its stiffness and insulates the core from the influence of changing external conditions. An integrally formed catheter body can generally be constructed from one or more materials that experience a change in stiffness due to a combination of the ambient conditions in the external catheter environment and in the lumen.

### BRIEF DESCRIPTION OF THE DRAWINGS

The forgoing and other features of the invention are hereinafter discussed with reference to the figures.

FIG. 1 is a cross-sectional view of an exemplary catheter in accordance with the invention.

FIG. 2 is a schematic representation to illustrate external influences on the material of the catheter.

FIGS. 3 to 5 are respective representations illustrating the change in the stiffness of the catheter under different ambient conditions.

### DETAILED DESCRIPTION

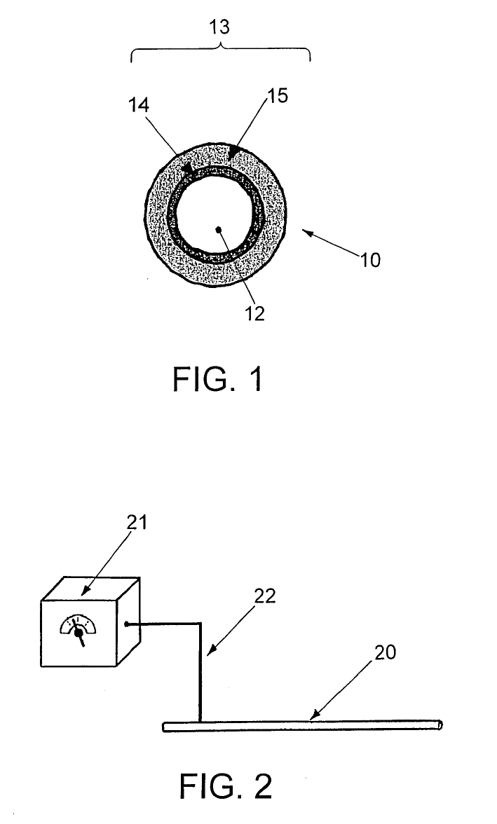

FIG. 1 schematically shows a cross-section of an exemplary catheter 10 in accordance with the present invention. The catheter 10 encloses a lumen 12 in which a fluid (for example a drug) is transported. The lumen 12 is surrounded by a catheter body 13 that includes a core 14 and an outer covering 15. In one example, the covering 15 comprises a material that changes its stiffness in accordance with changes in the ambient conditions, e.g., the material becomes more flexible when an external influence acts on it. The covering material 15 can be a silicone hydrogel that becomes more flexible or softer when it comes into contact with water.

The core 14 may be a Teflon coating that insulates the substance in the lumen 12 from the influence of the external conditions and protects the covering material 15 from the influence of the substance in the lumen 12. The catheter 10 in FIG. 1 also can be made flexible by external influences other than by a concentration of water, and one example is shown in FIG. 2. FIG. 2 shows a catheter 20 and a device 21 that generates a magnetic field or an electrical signal (current/voltage). A line 22 is intended to schematically indicate that the ambient conditions generated by the device 21 act on the catheter 20. The line of effect 22 can for example reflect the effect of a magnetic or electromagnetic field, or can reflect conveying a current or generating a voltage.

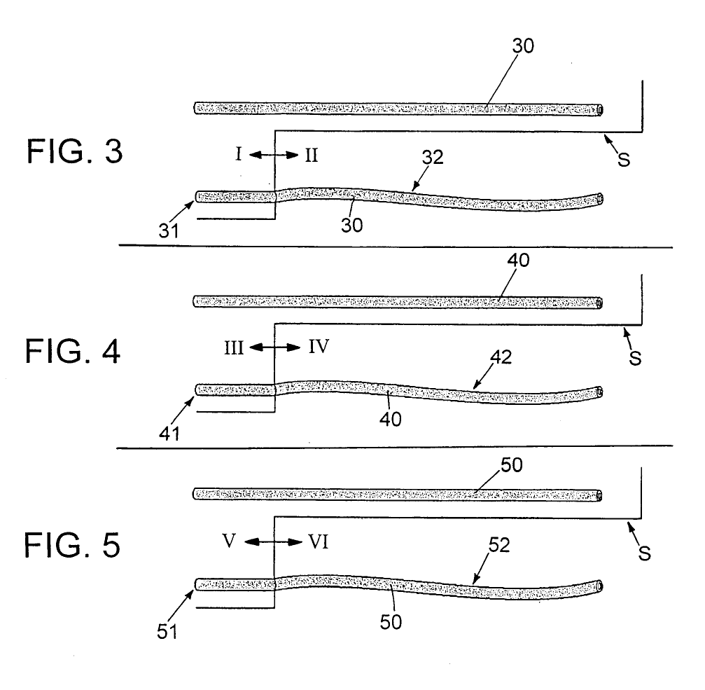

FIGS. 3 to 5 each show catheters in their initial state (top) and in a state in which they have been made flexible (bottom). In each of FIGS. 3 to 5, a line S indicates a line of separation that schematically indicates that different ambient conditions prevail on the two sides of the line. The ambient conditions are indicated by Roman numerals I to VI.

The top representation in FIG. 3 shows a catheter 30 in its rigid state. On the side I of the line of separation S, ambient conditions prevail which leave the material of the catheter 30 rigid and, in the case of FIG. 3, such an ambient condition I is a particular pH value outside a patient's body. On the other side II of the line of separation S, a pH value prevails that is different, for example a somewhat lower pH value, such as can occur in body liquids. The lower representation in FIG. 3 shows how a part 31 (the proximal end of the catheter 30), that is still under ambient conditions I, remains rigid, as shown the linear profile. On the side where the ambient condition II prevails, the catheter 30 has become flexible, as shown by a bent distal portion 32. The catheter 30 need not automatically bend under the influence of the ambient condition II; the bending merely serves to indicate the flexibility in the drawing. The distal portion 32, e.g., the region of the catheter that remains in a patient's brain, can bend due to its increased flexibility after it has been inserted in its rigid state under the ambient conditions I.

FIGS. 4 and 5 correspond in their essential representation to FIG. 3, and each figure shows a catheter 40 and a catheter 50 respectively. Catheters 40 and 50 both remain stiff under the ambient conditions III and V, respectively, but become flexible under ambient conditions IV and VI, at least in their respective distal portions 42 or 52. The regions beyond the line of separation S, e.g., the proximal portions 41 and 51, respectively, remain rigid.

The exemplary embodiments in FIGS. 4 and 5 differ in their ambient conditions. For example, ambient condition III represents a particular chemical environment, such as an air environment. The chemical environment IV may be an environment in which a certain humidity contacting the outer surface of the catheter prevails or in which specific chemical substances are present, wherein these influences make the distal portion 42 more flexible than it was in the initial state under the chemical conditions III.

This applies analogously to the representation in FIG. 5, wherein on the side of the line of separation S indicated by V, a different magnetic or electrical environment prevails than on the side VI. One example is a magnetic field not present in the environment V, while in the environment VI, a magnetic field has been generated by a corresponding magnetic field generator (not shown). The magnetic field causes a distal portion 52 of a catheter 50 to become flexible, while a proximal part 51 of the catheter 50, which is outside the magnetic field, remains rigid.

Under ambient conditions I, III and V, the catheter can be stereotactically placed as a rigid body. After a certain adapting time, the stiffness of the catheter material changes and under ambient conditions II, IV and VI, the catheter is flexible enough to follow the movements of the tissue.

Although the invention has been shown, and described with respect to a certain preferred embodiment or embodiments, it is obvious that equivalent alterations and modifications will occur to others skilled in the art upon the reading and understanding of this specification and the annexed Figures. In particular regard to the various functions performed by the above described elements (components, assemblies, devices, software, computer programs, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A catheter for administering a substance into a body tissue, comprising:
a lumen;
an elongated catheter body surrounding said lumen;
wherein at least a portion of the catheter body comprises a material that changes stiffness in response to changes in ambient conditions.

2. The catheter according to claim 1, wherein the stiffness of the material is reduced in response to the ambient conditions.

3. The catheter according to claim 1, wherein the material changes stiffness in response to physical or chemical influencing factors.

4. The catheter according to claim 1, wherein the material changes stiffness in response to changes in voltage and/or electrical current.

5. The catheter according to claim 1, wherein the material changes stiffness in response to magnetic field changes.

6. The catheter according to claim 1, wherein the material changes stiffness in response to pH values.

7. The catheter according to claim 1, wherein the material changes stiffness in response to temperature.

8. The catheter according to claim 1, wherein the material changes stiffness in response to a concentration of water.

9. The catheter according to claim 1, wherein the material changes stiffness in response to a concentration of ions.

10. The catheter according to claim 1, wherein the material changes stiffness in response to a concentration of a chemical substance or compound.

11. The catheter according to claim 1, wherein the ambient condition is a bodily ambient condition.

12. The catheter according to claim 1, wherein the material changes stiffness in response to a property of the substance to be administered.

13. The catheter according to claim 1, wherein the catheter body comprises an inner core formed around the lumen, and an outer covering formed around the inner core, wherein the inner core and outer covering are comprised of different materials.

14. The catheter according to claim 13, wherein the inner core and the outer covering are formed as an integral unit.

15. The catheter according to claim 13, wherein the covering comprises a material that changes stiffness when external ambient conditions of the covering are altered, and the core comprises a material that does not change stiffness and insulates the covering from the influence of the substance to be administered.

16. The catheter according to claim 13, wherein the core comprises a material that changes stiffness in the presence of the substance to be administered, while the covering comprises a material that does not change stiffness and insulates the core from the influence of external ambient conditions.

17. The catheter according to claim 1, further comprising a catheter body made of one or more materials that experience a change in stiffness in response to a combination of changes in the ambient conditions in the external catheter environment and in the lumen.
